(19) United States
(12) Patent Application Publication
(10) Pub. No.: US 2017/0135789 A1
(43) Pub. Date: May 18, 2017

(54) METHOD FOR PRODUCING TOOTH REPLACEMENTS AND AUXILIARY PARTS

(71) Applicant: BEGO Bremer Goldschlägerei Wilh. Herbst GmbH & Co. KG, Bremen (DE)

(72) Inventors: Haig Dolabdjian, Feldkirchen (DE); Roland Strietzel, Lilienthal (DE)

(21) Appl. No.: 15/422,194

(22) Filed: Feb. 1, 2017

Related U.S. Application Data
(63) Continuation of application No. 13/316,062, filed on Dec. 9, 2011, now abandoned.
Application No. 13/316,062 is a continuation of application No. 12/578,371, filed on Oct. 13, 2009, now abandoned.
Application No. 12/578,371 is a continuation of application No. 10/976,734, filed on Oct. 29, 2004, now abandoned.
Application No. 10/976,734 is a division of application No. 10/146,610, filed on May 14, 2002, now abandoned.
Application No. 10/146,610 is a continuation-in-part of application No. 10/081,039, filed on Feb. 19, 2002, now abandoned.
Application No. 10/081,039 is a continuation of application No. 09/481,306, filed on Jan. 11, 2000, now abandoned.

(30) Foreign Application Priority Data
Jan. 19, 1999 (DE) 19901643.7

(51) Int. Cl. A61C 13/00; A61C 5/73; A61C 13/09; A61K 6/04; B28B 1/00; B33Y 10/00; B33Y 80/00; A61C 5/77; A61C 13/20

(52) U.S. Cl. CPC A61C 13/0018; A61C 5/77; A61C 13/0004; A61C 5/73; A61C 13/09; A61C 13/20; B28B 1/001; B33Y 10/00; B33Y 80/00; A61K 6/04

(57) ABSTRACT

In a method for forming a dental part, a laser beam is guided over a powder layer of biocompatible material. The laser is guided by a computer controlled laser scanning system based on data representing the shape of the cross-section through the shaped body. The powder is substantially melted by the laser beam to form a layer in the shaped body, to build the shaped body entirely from layers of laser-melted material.

### CROSS-REFERENCES TO RELATED APPLICATIONS

This application is a continuation of U.S. patent application Ser. No. 13/316,062, filed Dec. 9, 2011, which is a continuation of U.S. patent application Ser. No. 12/578,371, filed Oct. 13, 2009, which is a continuation of U.S. patent application Ser. No. 10/976,734, filed Oct. 29, 2004, which is a divisional of U.S. patent application Ser. No. 10/146,610 filed May 14, 2002, which is a continuation-in-part of U.S. patent application Ser. No. 10/081,039 filed Feb. 19, 2002, which is a continuation of U.S. patent application Ser. No. 09/481,306 filed Jan. 11, 2000, which claims the benefit of priority from German Application No. 19901643.7, filed Jan. 19, 1999; the full disclosures of which are incorporated herein by reference in their entirety for all purposes.

### FIELD OF THE INVENTION

This invention relates to a method of forming a dental part and/or a tooth replacement part.

### BACKGROUND OF THE INVENTION

Tooth replacements in the form of crowns, bridges, inlays and the like frequently comprise complex molded bodies which must usually take account in each specific case of the spatial configuration of intact tooth parts (tooth stumps), entire teeth or parts of the jaw that have been lost, on the one hand, and the spatial situation in relation to adjacent and/or antagonistic teeth, on the other hand. In the prior art, such tooth replacement elements are produced in complex processes. The most widespread method is to produce the shaped bodies required—usually made of precious-metal or base-metal alloys, as well as pure metals—in a multi-step impression and casting process.

Computer-controlled milling of such shaped bodies out of the solid material has become known. This method inevitably leads to considerable waste that has to be reprocessed at great effort and expense.

### SUMMARY OF THE INVENTION

The objective of the invention is to provide another, more advantageous way of producing such shaped bodies (and auxiliary dental parts required in implantology) that provides flexibility in manufacturing dental parts of different shapes, but which reduces the amount of waste and results in a strong dental part.

A method in accordance with the principles of the invention includes a method of making a shaped body for use as a dental part. The method comprises guiding a laser beam over a powder layer using a computer-controlled laser scanning system based on data representing the shape of a cross-section through the shaped body. The powder comprises a biocompatible material of grain size in the range from 0 μm to 50 μm, to create a layer in the shaped body. The method further comprises substantially melting the powder with the laser beam, and repeating the guiding and melting over successive powder layers using successive cross-sectional representative data so as to build the shaped body entirely from layers of laser-melted material.

In another embodiment of the present invention, a shaped dental part for use in a patient's mouth. The shaped dental part comprises a body formed from melted particles of biocompatible material, the body having a surface shaped to fit in the patient's mouth and having a density of up to 98% of the density of the biocompatible material. The particles having pre-melting sizes in the range 0 μm-50 μm, and having essentially equal proportions of alloy components in each particle.

### DETAILED DESCRIPTION OF THE INVENTION

The invention relates to a method that has become known in another field as “rapid prototyping” for producing complex tools or components as disclosed in U.S. Pat. No. 4,863,538 included herein by reference. According to said method, shaped bodies made of a sintering powder are built up in layers by exposing each layer successively to the energy of a laser beam that leads to local sintering, whereby the laser beam is guided over the respective powder layer by means of a computer-controlled system using data that represent the configuration of the shaped piece in this layer. As a result of supplying such energy, the powder elements affected in each case are superficially melted and form a fixed bond with each other and the underneath layer. Due to the precise focusing of the laser beam, the energy supply can be configured exactly—at relatively high density—and controlled in accordance with the stored spatial data of the shaped body required.

Conventionally, in a sintering process, compressed powdered material is heated to a temperature close to but not at melting, usually in a controlled-atmosphere furnace. This is done so that particles may bond by solid state bonding, but not melt. Such sintering increases both density and strength of the material, because compaction alone leads to both properties being low. The latter is also true with sintering without compaction (compressing) the powdered material, as is the case with the selective sintering process addressed before.

It has been found that, rather than selectively sintering metal powder by superficially melting the uncompressed material, a still considerably higher density of the finished product can be achieved by substantially entirely melting the powdered material, primarily metal. Quite surprisingly, such “selective melting” of the powder does not lead to uncontrolled flowing away of the material, probably because the cohesion forces suffice to keep the thin layer of material in place, even in its molten state.

Using this method of “selective melting”, the porosity of the resultant part is significantly less than what is achieved under conventional laser sintering. For example, densities achieved with the conventional selective laser sintering technique ranges from 70-80%, while the densities achieved through ceramic sintering techniques range from 60-70%. In contrast, the density of the resultant part using a method according to the invention may be greater than 98% of the density of the biocompatible material, and may be as high as 99.9% of the density of the biocompatible material. Thus, a dense, and therefore strong, part may be formed using the laser selective melting technique. This permits the resultant part to be made with the desired shape without using a mold, but the part is also more able to withstand the high stresses that result from biting and chewing.

Furthermore, the invention provides for a powder consisting of a biocompatible material of varying grain size between 0 μm and 50 μm. In contrast to current application of the selective laser sintering method for technical purposes, the invention thus ensures that the shaped body designed for dental purposes is compatible with human tissue (see Hoffmann-Axthelm, Lexikon der Zahnmedizin [Encyclopedia of Dental Medicine], 6th/11th edition, p. 97, and Reuling, Biokompatibilitat dentaler Legierungen [Biocompatibility of Dental Alloys]). The grain size distribution ensures the forming of dense layers with the advantage of minimal creation of cavities between the layer after melting which would be susceptible to bacteria cultures forming; in addition, it defines the size and fitting accuracy of the restoration.

While larger cross-sectional areas of the dental part to be produced, are impacted by the laser beam by oscillating it in one direction, and shifting the oscillating beam in a direction perpendicular thereto, as explained in U.S. Pat. No. 4,863,538 mentioned above, according to the invention the laser beam follows the contour of the wall to be produced within the cross-section of thin-walled areas.

Due to its certain degree of roughness, the surface of the shaped body produced in accordance with the invention is particularly well-suited for the frequently desired veneering process using ceramic or other materials, as is the case with crowns or bridges. Furthermore, because it is easy to influence the file on which the control process is based, it is possible to make corrections to the configuration of the shaped body that may appear desirable (with respect to the traced result) for a wide variety of reasons.

The powder preferably comprises an alloy with essentially equal proportions of the alloy components in each grain of powder. This provides a major advantage compared to the conventional production of shaped dental bodies from melted alloys, because there is no risk of segregation of the alloy components in the melt and/or in the shaped body after casting. In addition, the production of semi-finished products that are made of certain alloys and are particularly advantageous for dental purposes necessitates complicated and costly processes, such as suction casting and the like, whereas pulverization of such alloys is significantly less complex. However, whereas a melt produced from such a powder (for subsequent production of shaped cast bodies) is exposed for its part to the risk of segregation and thus non-homogeneity, a shaped body that is selectively melted according to the invention maintains its uniform distribution of alloy components.

A metal powder with the following composition has proved effective for use with the method according to the invention, whereby the method is not confined to said composition: Ni61, 4Cr22, 9M08, 8Nb3, 9Fe2, 5Mn0.4Ti0.1, where the alloy comprises 61.4% Ni, 22.9% Cr, 8.8% Mo, 3.9% Nb, 2.5% Fe, 0.4% Mn and 0.1% Ti.

What is claimed is:

1. A laser sintering process in which moulded bodies, including a dental prosthesis or dental auxiliaries, are constructed in layers from a sinterable powder such that each successive layer of the powder is exposed to energy of a laser beam resulting in local sintering;
wherein the guidance of the laser beam over the respective powder layer is subject to control by data which represents the configuration of the moulded bodies in the respective powder layer; and
wherein the sinterable powder consists of a biocompatible material of varying particle size between 0 μm and 50 μm.

2. The laser sintering process according to claim 1, wherein the dental prosthesis is a crown, a bridge, or an inlay.

3. The laser sintering process according to claim 1, wherein the sinterable powder consists of an alloy with substantially similar contents of the alloy constituents in each powder particle.

4. The laser sintering process according to claim 1, wherein the moulded bodies consist of the laser sintered powder.

5. The laser sintering process according to claim 1, wherein the energy supplied by the laser beam is controlled by stored spatial data of the moulded bodies.

6. The laser sintering process according to claim 1, wherein the moulded bodies are produced by a rapid prototyping process.

7. A method of making a shaped body for use as a tooth replacement part, the shaped body having a density, the method comprising:
guiding a laser beam over a powder layer using a computer-controlled laser scanning system based on data representing the shape of a cross section through the shaped body, the powder comprising a biocompatible material, the biocompatible material having a grain size up to about 50 μm, to create a layer in the shaped body;
substantially melting the powder with the laser beam; and
repeating the guiding and melting over successive powder layers using successive cross-sectional representative data so as to build the shaped body entirely from layers of laser melted material.

8. The method of claim 7, wherein the tooth replacement part is a crown, a bridge, or an inlay.

9. The method of claim 7, wherein the molten powder does not lead to uncontrolled flowing away so that the powder maintains the shape of each cross section through the shaped body.

10. The method of claim 7, wherein the shaped body has an average density of up to 98% of the density of the biocompatible material.

11. The method of claim 7, wherein the shaped body has an average density of up to 99.9% of the density of the biocompatible material.

12. The method of claim 7, wherein the powder comprises an alloy with substantially equal proportions of alloy components in each grain of the powder.

13. The method of claim 12, wherein the substantially equal portions of alloy components prevent segregation of the alloy components in the shaped body.

14. The method of claim 7, wherein the biocompatible material is a metal alloy.

15. The method of claim 7, wherein the biocompatible material is Ni61.4, Cr22.9, Mo8.8, Nb3.9, Fe2.5, Mn0.4, and Ti0.1.

16. The method of claim 7, wherein the molten powder substantially maintains the shape of each cross section through the shaped body.

17. An intermediate for being made into a shaped dental part for use in a patient's mouth, comprising:
a partial body comprising biocompatible material and having a surface shaped to fit in the patient's mouth; and
a layer of powder alloy disposed upon a surface of the partial body and comprising particles of the biocompatible material, the particles generally being of a predetermined density, having varying grain sizes in a range of about 0 μm to about 50 μm, and having essentially equal proportions of alloy components in each particle;
wherein the biocompatible material of the partial body has a density of not less than about 98% of the predetermined density of the particles.

18. The intermediate as recited claim 17, wherein the biocompatible material of the partial body has a density between 98% and 99.9% of the predetermined density.

19. The intermediate as recited in claim 17, wherein the biocompatible material is a metal alloy.

20. The intermediate as recited in claim 17, wherein the biocompatible material is 61.4% Ni, 22.9% Cr, 8.8% Mo, 3.9% Nb, 2.5% Fe, 0.4% Mn, and 0.1% Ti; and wherein the particle layer is of a thickness for forming a layer of cohesively maintained biocompatible material, when melted by a guided laser beam, to enlarge the partial body.
